(19) United States
(12) Patent Application Publication
(10) Pub. No.: US 2009/0281491 A1
(43) Pub. Date: Nov. 12, 2009

(54) INJECTION SYRINGE WITH AUTOMATICALLY RETRACTABLE NEEDLE

(75) Inventors: Peter Nilsson, Jonkoping (SE); Tomas Arnerdal, Bankeryd (SE); Johan Rinman, Aneby (SE)

Correspondence Address: WINSTON & STRAWN LLP;PATENT DEPARTMENT, 1700 K STREET, N.W., WASHINGTON, DC, 20006, US

(73) Assignee: MEDSAFE ASA, Olso (NO)

(21) Appl. No.: 12/303,099

(22) PCT Filed: Jun. 1, 2006
(86) PCT No.: PCT/IB2006/051755

§ 371 Date: Jul. 1, 2009

(51) Int. Cl. A61M 5/50; B29C 45/03

(52) U.S. Cl. 604/110; 264/250

(57) ABSTRACT

An injection syringe comprises a tubular housing, a hollow plunger disposed for reciprocating displacements, partly in the housing, between an advanced and a retracted position, an elongated retraction member arranged for being moved from an advanced to a retracted position in relation to the plunger with at least a part of the retraction member placed inside the plunger, and a spring acting with a spring power between the plunger and the retraction member; a hypodermic needle, displaceable between a first position, wherein it is releasable mounted in the housing and a part of the needle is protruding from the housing, and a second position, wherein the needle is retracted into the housing or the plunger; a releasable retainer arrangement acting between the plunger and the retraction member for retaining the retraction member in its advanced position in relation to the plunger, a releasing mechanism for releasing the retainer when the plunger is in or close to its advanced position, and a coupling for connecting the retraction member and the needle during or immediately after releasing the retainer.

### FIELD OF THE INVENTION

The invention relates to an injection syringe and to a method for producing this syringe.

The injection syringe comprises a tubular housing, a hollow plunger disposed for reciprocating displacements, partly in the housing, between an advanced and a retracted position, an elongated retraction member arranged for being moved from an advanced to a retracted position in relation to the plunger with at least a part of the retraction member placed inside the plunger, and at least one spring acting with a spring power between the plunger and the retraction member.

### BACKGROUND ART

From the applicant's international patent application PCT/EP2004/005997, which is incorporated in the present patent application by reference, is known a disposable injection syringe comprising a piston, which is reciprocatingly mounted in a housing. The piston is connected with a piston rod for manually operating the piston. The known syringe moreover comprises a coupling for at the end of an injection stroke connecting the piston with a hypodermic injection needle protruding from the housing. The needle is then retracted into the housing by manually retracting the piston. The needle is, in retracted position, tilted in such way that it is prevented from protruding from the housing once more.

It is by means of this known disposable injection syringe effectively prevented that bacteria, which may adhere to a protruding hypodermic injection needle of an already used syringe, accidentally or by re-use infect other persons.

The U.S. Pat. Nos. 5,385,551, 5,578,011 and 6,090,077 disclose disposable injection syringes arranged for automatically retracting the injection needle at the end of an injection stroke. The needle is attached to a needle holder releasable mounted in the tip of the housing of the syringe loaded by a pre-stressed spring placed in the tip too. A retainer arrangement serves to keep the needle holder in position. The injection takes place by pushing a plunger forward in the housing. The plunger is formed with an internal cavity closed by a resilient, dislodgeable stopper. The plunger activates, at the end of the injection stroke, the retainer arrangement which then releases the needle holder, after which the spring power of the spring presses the needle holder against the stopper in the plunger by the spring power of the spring. This pressure dislodges the stopper thereby allowing the needle holder with the needle to be forced into the cavity of the plunger during the expansion of the spring.

The spring of these known syringes has a relatively little diameter and a relatively short length in compressed state. The capacity of the spring is therefore very limited as regards being able to present the necessary power for forcing the needle holder with the needle together with the stopper all the way until the needle is fully retracted in the housing and/or the cavity of the plunger and the syringe therefore safely can be disposed.

Another drawback consists in that the power for dislodging the stopper tends to force the needle holder with the needle in the direction out of the hosing instead of into the housing with the risk of getting jammed in the tip of the housing whereby the desired retracting of the needle might be unreliable.

Since the plunger need to be displaced an empty distance during the dislodging of the stopper, the syringe cannot be emptied to a sufficient extent during an injection stroke. The known syringes are therefore relatively uneconomical to use. A similar syringe is known from U.S. Pat. No. 5,389,076. The spring is, in this case, acting on the stopper instead of the needle holder allowing the spring to be more appropriately dimensioned. The needle holder and the stopper are however preliminarily connected with each other thereby increasing the risk of the desired retracting of the needle being unreliable. Also this syringe suffers from the problem of not emptying the syringe sufficiently of fluid during an injection stroke.

Owing to the fact that plunger needs to be displaced an empty distance in the housing of the syringe during dislodging, the stoppers will, in the above mentioned four U.S. patents, all function with a relatively large inaccuracy when injecting a portion of fluid, e.g. medicine, into a patient.

Said prior art syringes moreover have a relatively complicated and therefore expensive construction.

The above-mentioned disadvantages of the prior art syringes are according to the present invention remedied by,

in a first aspect of the invention providing an injection syringe of the kind mentioned in the opening paragraph, with which the hypodermic needle more safely than hitherto known will be retracted automatically into the syringe at the end of an injection stroke,
in a second aspect of the invention providing an injection syringe of the kind mentioned in the opening paragraph, in which an already retracted needle is effectively secured against protruding once more from the housing of the syringe,
in a third aspect of the invention providing an injection syringe of the kind mentioned in the opening paragraph, in which the needle is protected against being stressed by a force acting in the opposite direction of the force for retracting the needle,
in a forth aspect of the invention providing an injection syringe of the kind mentioned in the opening paragraph, in which the fluid, e.g. medicine, can be dosed more accurately than hitherto known,
in a fifth aspect of the invention providing an injection syringe of the kind mentioned in the opening paragraph, in which the syringe can be emptied of fluid, e.g. medicine, to a larger extent than hitherto known,
in a sixth aspect of the invention providing an injection syringe of the kind mentioned in the opening paragraph, which has a simple and inexpensive construction,
in a seventh aspect of the invention providing an injection syringe of the kind mentioned in the opening paragraph, which is well suited for mass production, and
in an eight aspect of the invention providing an injection syringe of the kind mentioned in the opening paragraph, which at least only to a less extent can be affected by shocks coming from the initial retracting process.

### SUMMARY OF THE INVENTION

The novel and unique features of the invention consist in the fact that the injection syringe further comprises a hypodermic needle which is displaceable between a first position, wherein it is releasable mounted in the housing and a part of the needle is protruding from the housing, and a second position, wherein the needle is retracted into the housing and/or the plunger, a releasable retainer arrangement acting between the plunger and the retraction member for retaining the retraction member in its advanced position in relation to the plunger, a releasing means for releasing the retainer when the plunger is in or close to its advanced position, and a coupling for connecting the retraction member and the needle during or immediately after releasing the retainer.

The syringe of this construction is safe, accurate and economic to use and can be disposed without any risk of other persons being infected by bacteria adhered to the needle. The syringe can moreover be mass-produced in a very economical way.

The coupling of the invention is arranged for transmitting pulling power only between the retraction member and the hypodermic needle thereby advantageously avoiding the risk of blocking the retracting of the needle at the end of the injection stroke by acting on the needle with a force in the opposite direction of the retracting direction.

According to the invention the coupling can comprise a first and second coupling part, whereby the first one can have a hook and the second one a ring arranged for allowing the hook to pass through the ring in the direction of the needle but not in the opposite direction.

The hook will at the end of an injection stroke thereby easily be able to pass the ring but is afterwards prevented from disengaging the ring whereby a traction force can be transmitted from the retraction member to the needle for retracting this into the housing and/or the hollow plunger.

In an advantageous embodiment according to the invention the hook can be arranged at the end of a protruding pin of the retraction member, and the ring can moreover be connected with a holder for the needle by means of at least one strap having a larger length than the length of the hook whereby it is effectively prevented that the needle holder with the needle can be acted on with a force in the opposite direction than the retracting direction.

According to the invention the releasing means can comprise a first releasing stop on the housing co-operating with a second releasing stop on the retraction member for preventing further advancing of the retraction member when the plunger is pushed forward in the housing to an intermediate position situated at a distance from the advanced position of the plunger. The retainer arrangement acting between the plunger and the retraction member is released when pushing the plunger further forward from said intermediate position, as the retainer arrangement is formed with a resistance against axial loadings which is lower than the reaction force acting between the first and second releasing stop when pushing the plunger forward with a predetermined power.

The retainer arrangement can, in one embodiment according to the invention, comprise a first retraining part on the plunger and a second retraining part on the retraction member, which retraining parts can be deformed in relation to each other to such an extent that they will pass each other when pushing the plunger past its intermediate position by means of sufficient power thereby releasing the retraction member.

In an appropriate embodiment of the invention the first retraining part can be equipped with at least one first projection and the second retraining part with at least one second projection, whereby the first and second projections overlap each other radially and abut against each other influenced by the spring power of the spring. The projections can be helpful for, by acting on the plunger with relatively little force, providing the necessary deformation of the first and/or the second retaining parts for releasing the retainer arrangement. Said deformation can result in the retaining parts breaking.

In a preferred embodiment of the invention the tubular housing can comprise a first tubular housing part accommodating the plunger, a second tubular housing part having a smaller diameter than the first one, and a transverse third housing part connecting the first and second housing parts.

In a simple and effective embodiment of the invention the first releasing stop can then be formed on the third housing part while the second releasing stop is an opposite shoulder on the retraction member.

According to the invention the plunger at its end portion can be equipped with a gasket for sealing the hollow plunger in relation to the first tubular housing part, which gasket advantageously can be arranged for simultaneously sealing the hollow plunger in relation to the retraction member in the advanced position of this. The gasket thereby enables the plunger to expel the fluid in the syringe through the hypodermic needle while the plunger is pushed forward from its retracted to its advanced position.

An important part of the invention consists in the gasket being able to be compressed in such way that it will abut at least a part of the inner side of the third housing part while pushing the plunger forward in the first housing part from at least the intermediate position of the plunger thereby advantageously securing that as much of the fluid in the syringe as possible is utilized. The gasket can e.g. be made of an elastomer.

Retracting the injection syringe according to the present invention from a patient, which have been subjected to an injection, can be made in at least two different ways.

The first way is to withdraw the needle from the patient and subsequently activate the retraction operation of the needle. Alternatively, the retraction operation may be initiated already while the needle is located inside the patient. In the latter case the shock absorber means effectively serves for a gentle and comfortable retraction of the needle out of the patient with a minimum of sensation of pain.

When the hypodermic needle is withdrawn from a patient, a length of the needle is contaminated with blood. If a blood droplet is left on the tip of the needle, at least some of said droplet could be lost to the surrounding environment if retraction of the needle is done without great consideration to the manipulation of the syringe after injection. If the retraction is done too fast at least a part of the droplet is thrown off the tip of the needle before the needle is fully retracted into the cavity of the plunger. As a result at least some of the droplet is dispersed and aerified outside the syringe to contaminate the environment with airborne blood residues thereby enhancing overall danger of infection.

To avert this problem the injection syringe can according to the invention in an advantageous embodiment comprise a shock absorber means adapted for, at least in the main, slow down the retraction operation.

The shock absorber means advantageously serves for preventing the injection syringe from being affected by shocks during retraction of the hypodermic needle into the cavity of the plunger.

Due to the gentle and controlled retraction of the needle the shock absorber means secures the patient against being injured during the retracting operation. The syringe does not shake the hand holding the syringe, thereby reducing the risk of that the needle unintentionally hits and harms somebody before fully retraction is obtained.

The shock absorber means can in a simple and effective embodiment consists of a blind hole extending into the retraction member from a terminal surface opposite the head on the retraction member and of a piston which is slidable placed in the blind hole and is connected to the fourth retraction member.

This construction forms a pneumatic shock absorber in which the force needed for retracting the hypodermic needle will slide the piston into the direction of the mouth of the blind hole, whereby negative pressure is generated in the blind hole. The negative pressure generated in this way will form a reaction force counteracting the retraction force. Therefore, at the beginning of the retraction operation the resultant of said two forces will be so little that it hardly can affect the injection syringe.

An elastic strap can in another embodiment according to the invention be attached to the blind hole at one end and to the piston at the other end. The elastic strap is stretched when the retraction force slides the piston into the direction of the mouth of the blind hole whereby the elastic force of the stretched strap will counteract the retraction force. The resultant of the two forces therefore also in this case will at the beginning of the retraction operation be so little that it hardly can affect the injection syringe.

In a preferred embodiment according to the invention can the shock absorber means comprise that the clearance between the head of the retraction member and the hollow plunger taper off against at least one of the end regions of the cavity of the plunger. Thereby is obtained that the retraction stroke at the beginning proceeds very moderate and gentle, then rapidly and finally very moderate again, with the advantageous result that the retraction operation will proceed in very moderate manner but still very fast.

The syringe of the invention can be produced in a very economical way by simultaneously injection moulding the hollow plunger and the elongated retraction member in such way that the first retraining part on the plunger and the second retraining part on the retraction member are integral moulded.

### BRIEF DESCRIPTION OF THE DRAWINGS

FIG. 1 is a cross section along the axis of the injection syringe of the invention with the hollow plunger positioned in a starting position for the injection operation,

FIG. 2 shows the same syringe with the plunger positioned in an intermediate position in relation to the retraction member in which this are ready to be released,

FIG. 3 shows the same syringe during the releasing of the retainer arrangement,

FIG. 4 shows the same syringe with the plunger positioned in an advanced position, in which at least the major part of the fluid in the syringe has been expelled through the hypodermic needle of the syringe,

FIG. 5 shows the same syringe with the plunger positioned in a retracted position, in which the hypodermic needle is retracted into the syringe,

FIG. 6 is a cross section along the axis of a fragment of the syringe of FIG. 1-5 showing on a greater scale a first embodiment of the retainer arrangement with the plunger positioned in the intermediate position, in which the retraction member are ready to be released, and also the coupling for coupling the retraction member and the hypodermic needle of the syringe together after the retraction member has been released,

FIG. 7 shows the fragment of FIG. 6 in which the retainer arrangement is being released,

FIG. 8 shows the fragment of FIG. 6 in which the retainer arrangement has been released and the retraction member and the hypodermic needle of the syringe are ready to be coupled together,

FIG. 9 shows the fragment of FIG. 6 in which the retainer arrangement has been released and the retraction member and the hypodermic needle of the syringe have been coupled together,

FIG. 10 is a cross section along the axis of a fragment of the syringe of FIG. 1-5 showing on a greater scale a second embodiment of the retainer arrangement ready to be released and the coupling for coupling the retraction member and the hypodermic needle of the syringe together after releasing of the retainer arrangement,

FIG. 11 shows the fragment of FIG. 10 in which the retainer arrangement is being released,

FIG. 12 shows the fragment of FIG. 10 in which the retainer arrangement has been released and the retraction member and the hypodermic needle of the syringe are ready to be coupled together,

FIG. 13 shows an embodiment of the injection syringe of FIG. 1-12 but equipped with a first embodiment of a shock absorber seen in one phase,

FIG. 14 shows the same but with the shock absorber seen in another phase,

FIG. 15 shows an embodiment of the injection syringe of FIG. 1-12 equipped with a second embodiment of a shock absorber seen in one phase,

FIG. 16 shows the same but with the shock absorber seen in another phase, and

FIG. 17 shows, somewhat exaggerated, a fragment of another embodiment of a plunger to the injection syringe shown in FIG. 1-12.

### DETAILED DESCRIPTION OF THE INVENTION

In the description that follows it is assumed that the injection syringe of the invention is used for injecting a medicine into the body of a patient. The syringe can be a syringe prefilled with the medicine or an empty syringe which is filled with the medicine when the injection operation is to take place.

The reference number 1 refers to the syringe in general. The main parts of the syringe have, in this case, a circular cross section but may in other embodiments have other cross sections. Like parts are referred to by the same reference numerals.

The syringe comprises an elongate tubular housing 2, consisting of a first tubular housing part 3 and a second tubular housing part 4, which preferable is having a smaller diameter than the first tubular housing part. Said two housing parts are merging into each other via a traversal third housing part 5.

The second housing part 4 consists of a muff 6 extending from the transverse third housing part 5 and a cap 7 with a through hole 8 in its end portion 9 for introduction of a hypodermic needle 10 into the cap.

The muff 6 and the cap 7 could within the scope of invention be formed in any appropriate way. In practice is it, however preferred to form the muff and cap 6,7 as conical fittings with a 6% (Luer) taper according to European Standard No. EN 20594-1:1993/A1 or International Standard ISO 594-2:1998(E).

An elongate plunger 11 is disposed for reciprocating displacements in the first tubular housing part 3 with a part of the plunger projecting from said house part. Inside the plunger is formed with an elongate cavity 12 and is at the end portion of the plunger equipped with a casket 13 for sealing the plunger in relation to the first housing part 3.

An elongated retraction member 14 is arranged for being moved from an advanced to a retracted position in relation to the plunger. At least a part of the retraction member is placed in the cavity 12 of the plunger.

In this case the retraction member has a first retraction member part 15, a second retraction member part 16, a third retraction member part 17 and a fourth retraction member part 18 which at the end has a hook 19. The first retraction member part 15 has a larger diameter than the second retraction member part 16 and is merging into this via a first shoulder 20. The second retraction member part 16 has a larger diameter than inner diameter of the muff 6 and of the third retraction member part 17 and is merging into this via a second shoulder 21. The third retraction member part 17 has a larger diameter than the fourth retraction member part 18 and is merging into this via a third shoulder 22.

The gasket 13 for sealing the plunger in relation to the first tubular housing part 3 of the tubular housing 2 is arranged for also sealing the plunger in relation to the second retraction member part 16 of the retraction member 14. The gasket and the retraction member are thereby making the plunger tight for, during an injection stroke, effectively and precisely being able to expel the medicine in the syringe through the protruding hypodermic needle of the syringe in an exactly measured quantity.

The gasket is able to be compressed in such way that it will abut at least a part of the inner side of the third housing part while pushing the plunger forward in the first housing part from at least the intermediate position of the plunger thereby advantageously securing that as much of the fluid in the syringe as possible is utilized. The gasket can e.g. be made of an elastomer.

A spring 23 is acting between a collar 24 of a head 25 on the retraction member and an inwardly facing shoulder 26 on the plunger.

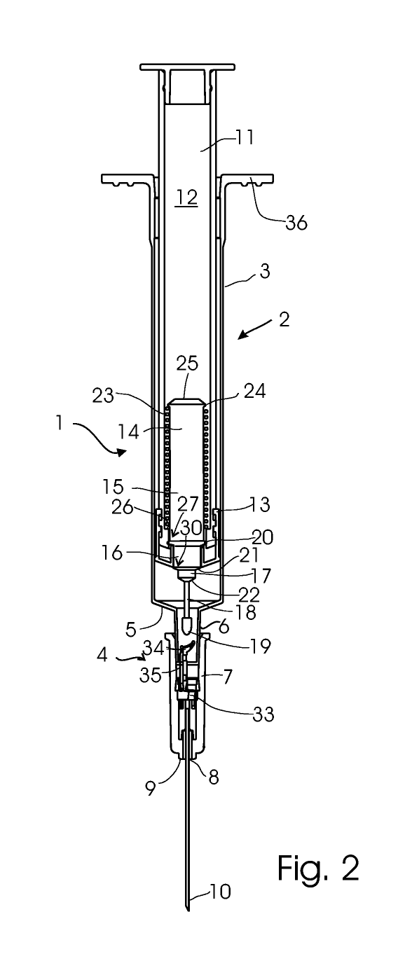

In the position of the plunger shown in FIG. 2 the spring 23 is pre-stressed, that means that the spring is acting on the retraction member with a spring power trying to force the retraction member to the rear in the plunger. The retraction member is however retained in said position by means of a releasable retainer arrangement 27 consisting of a first retainer part 28 on the plunger and a second retainer part 29 on the retraction member.

A releasing means 30 is arranged for releasing the retainer arrangement when the plunger is pushed forward from the position shown in FIG. 2. The releasing means comprises a first releasing stop 31 on the housing and a second releasing stop 32 on the retraction member. The first releasing stop is an inner part of the inner side of the transverse third housing part 16 and the second releasing stop is an outer part of the second shoulder 21 of the retraction member.

The hypodermic needle 10 is fastened in a needle holder 33, which is releasable mounted in the cap 7. The needle holder is at a distance from the needle holder connected to a ring 34 by means of, in this case, a single strap 35.

The hook 19 on the fourth retraction member part 18 of the elongate retraction member 14 forms a coupling together with the ring 18 as the hook and the ring are formed in such way that the hook is allowed to pass through the ring in the direction of the needle holder but not in the opposite direction.

Said coupling can transmit pulling power only between the retraction member 14 and the needle holder 33 via the strap 35 which in an advantageously embodiment cannot resist compressive forces. Moreover there still is a distance between the hook and the needle holder when the plunger and retraction member both are in their advanced positions thereby advantageously preventing that the needle holder with the needle is pushed more or less out of the cap 7 with the risk that the retracting of the needle holder is blocked.

The injection syringe of the invention is functioning in the following way.

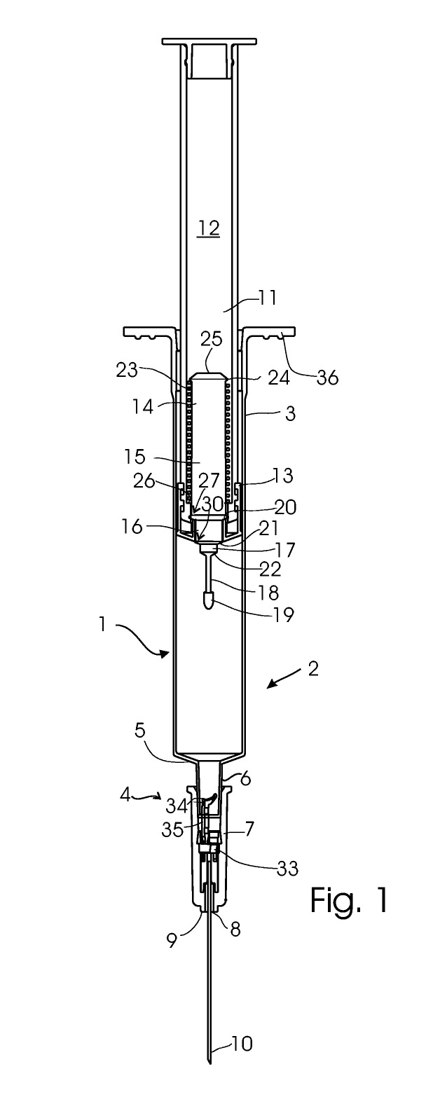

In FIG. 1 the hollow plunger of the syringe is positioned in a starting position for the injection of a medicine (not shown) into a patient (not shown).

The introduction end of the first tubular housing part 3 of the tubular housing 2 is equipped with a collar 36 for holding the housing with normally two fingers (not shown) while the hollow plunger 11 is pushed into the housing by the pressure of a third finger (not shown) for thereby expelling the medicine through the hypodermic needle.

In FIG. 2 the plunger has, in this way, been pushed into an intermediate position, in which the releasing means are ready to release the retainer arrangement.

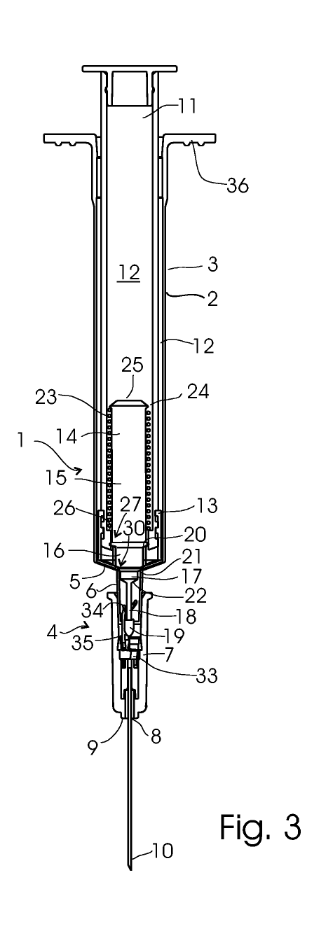

The retainer arrangement is in FIG. 3 now going to be released by pushing the plunger past the intermediate position.

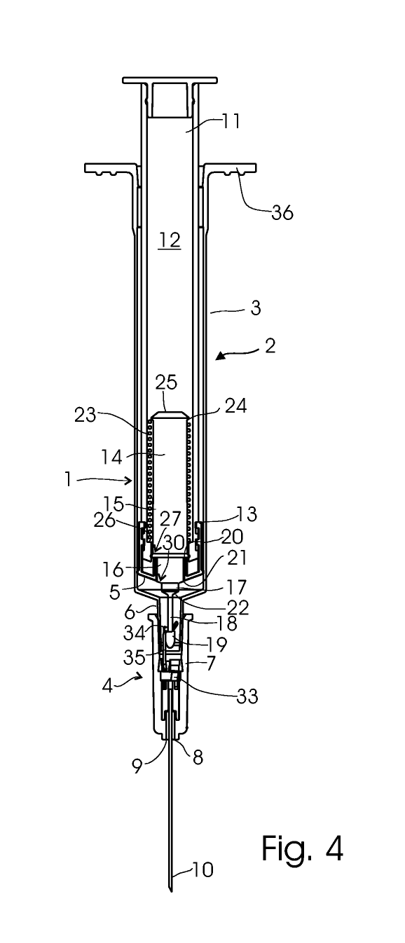

In FIG. 4 the plunger has been pushed into an advanced position in which at least the major part of the fluid in the syringe has been expelled through the hypodermic needle of the syringe. The retainer arrangement has at the same time been fully released.

Releasing the retainer arrangement for retaining the elongated retraction member 14 in its advanced position in the hollow plunger 11 results in the spring power of the pre-stressed spring 23 forcing the retraction member into the hollow plunger. The needle holder will simultaneously be retracted into the hollow plunger as the retraction member and the needle holder are coupled together immediately after releasing of the retraction member.

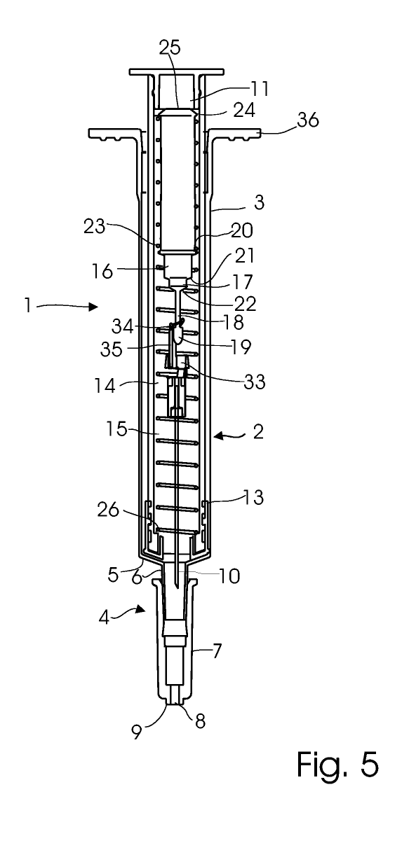

FIG. 5 shows that the needle is now fully retracted in the tubular hoses and the hollow plunger thereby effectively preventing the bacteria that may adhere to the needle after the injection has taken place from accidentally or by re-use infecting other persons.

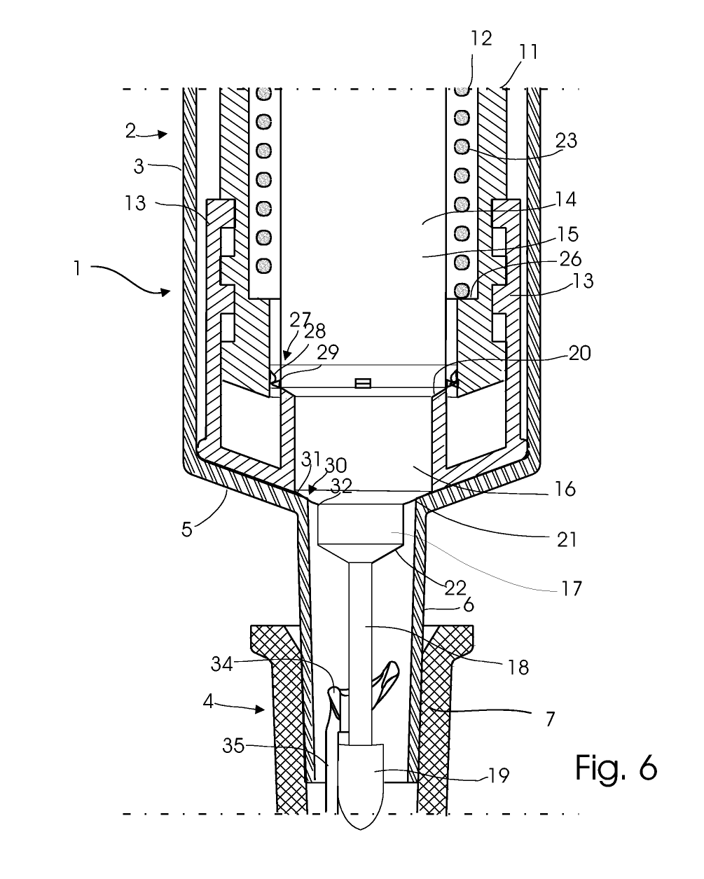

FIGS. 6-9 show on a greater scale a fragment of the syringe of the invention with a first embodiment of the retainer arrangement of the invention.

The releasable retainer arrangement 27 consists in this case of a ring-formed first projection 28 in the hollow plunger and in this case four projections 29 on the first retraction part 14 placed close to the first shoulder 20.

It is noted that the first ring-formed projection instead can be formed on the first retraction part while the four second projections can be formed in the hollow plunger and also that the number of the second projections can be different from number of four.

In FIG. 6 the plunger is in its intermediate position where the retainer arrangement is ready to be released. As can be seen the component and the second projections 29 overlap each other and furthermore abut each other influenced by the spring power of the pre-stressed spring.

The second projections 28 are arranged in such way that they can be deformed. The side of the first projection 28, abutting the four second projections 29 is furthermore inclined. Owing among other things to the fact that said side of the first projection is inclined the second projections 29 will deform when a sufficient force arises between the abutting first projection and second projections.

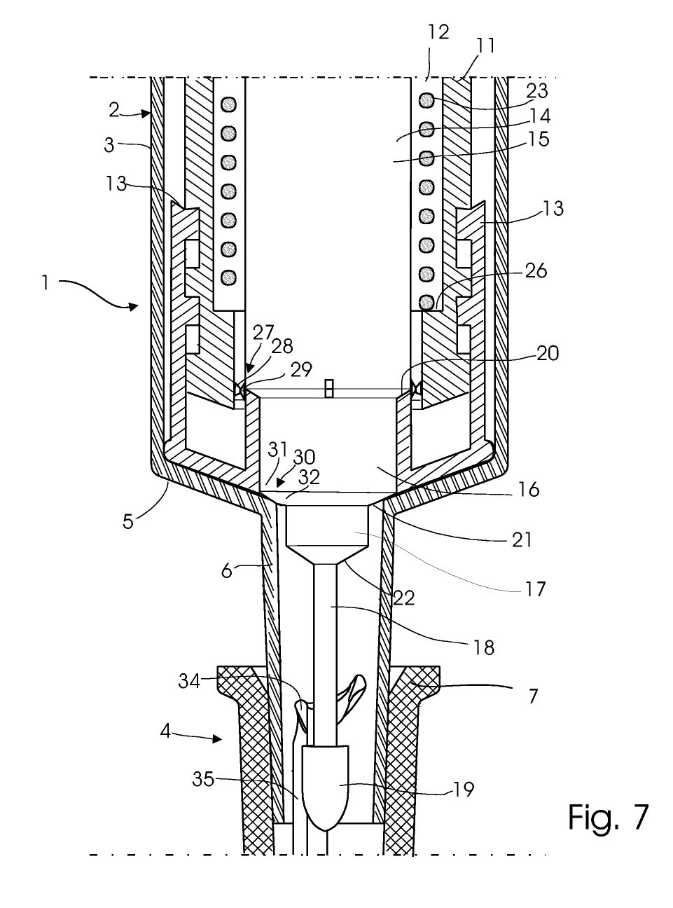

This force arises by further pushing the plunger a little distance into the housing from the intermediate position shown in FIG. 6 with a predetermined force since the second shoulder 21 of the retraction member 14 is engaging the transverse third housing part 5 in the intermediate position of the plunger thereby stopping further forward movement of the retraction member. The second projections 29 will then deform as seen in FIG. 7.

The second projections are in this deformed position able to pass the first projection and thereby release the retraction member.

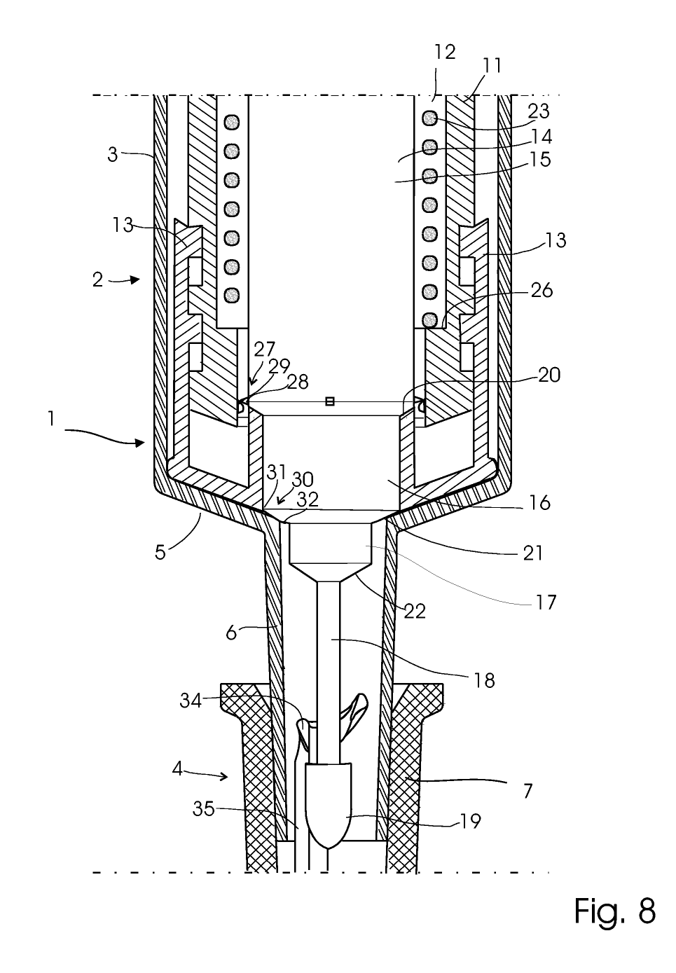

FIG. 8 shows the position of the retraction member immediately after the releasing of the retraction member where the hook 19 has now passed the ring 34 and the retraction member is ready to be retracted into the tubular housing and the hollow plunger influenced by the spring power of the spring.

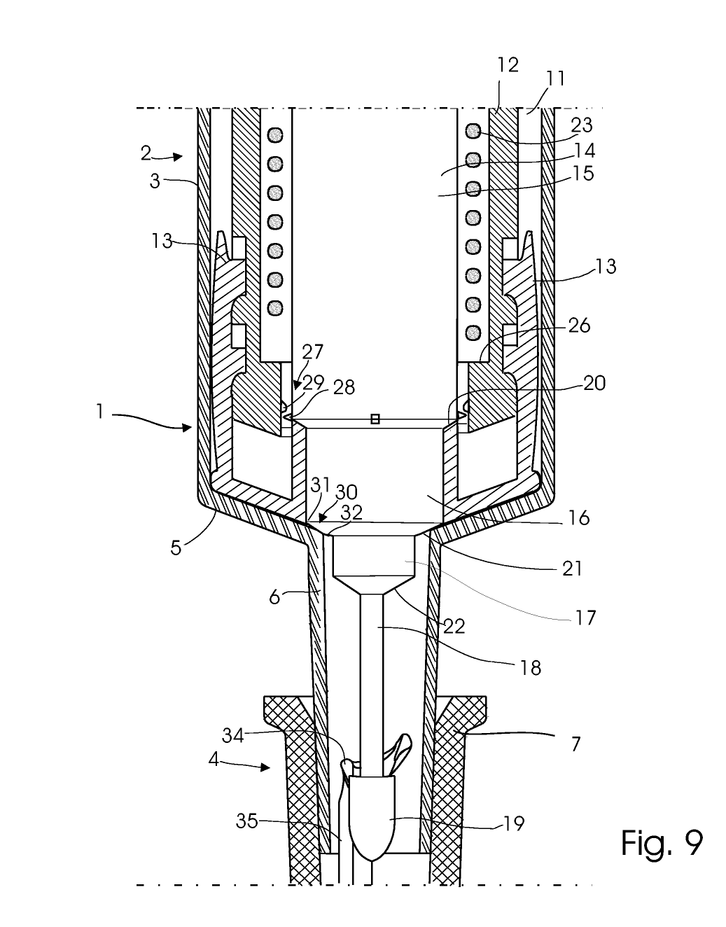

FIG. 9 shows the retraction member retracted so much into the hollow plunger that the hook and the ring couple together.

The retraction member and the needle holder are now connected in such way that the spring power of the spring acting on the retraction member will be transmitted to the hypodermic needle holder with the needle via the active coupling between the retraction member and the needle holder.

The needle holder with the hypodermic needle will therefore, during the continued retracting process, be pulled completely into the tubular housing and the hollow plunger. This situation is shown in FIG. 5.

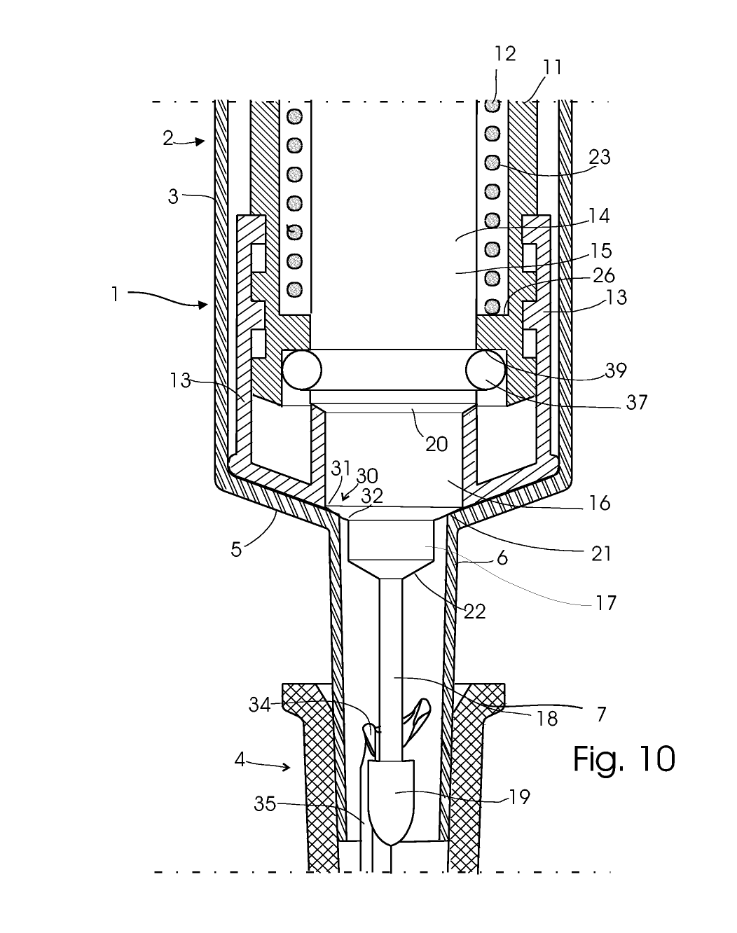

FIGS. 10-12 show on a greater scale a fragment of the syringe of the invention with a second embodiment of the retainer arrangement of the invention.

In FIG. 10 the plunger is in its intermediate position where the retainer arrangement is ready to be released. As can be seen, the releasable retainer arrangement consists of an elastic O-ring 37 placed in a groove 38 in the retraction member 14. A shoulder 39 on the hollow plunger 11 is simultaneously resting on the O-ring.

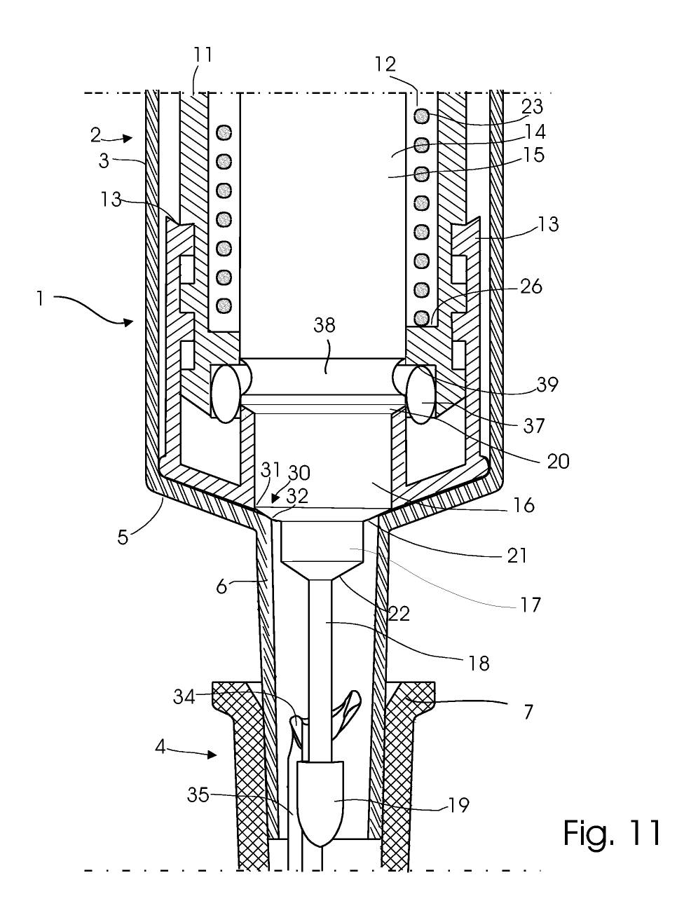

The hollow plunger will, when acted on by a predetermined force, be pushed forward a little distance further in the housing resulting in the shoulder 38 on the plunger forcing the O-ring 37 to leave the groove 38 in the retraction member 14, because the second shoulder 21 of the retraction member 14 is engaging the transverse third housing part 5 thereby stopping further forward movement of the retraction member. This situation is shown on FIG. 11

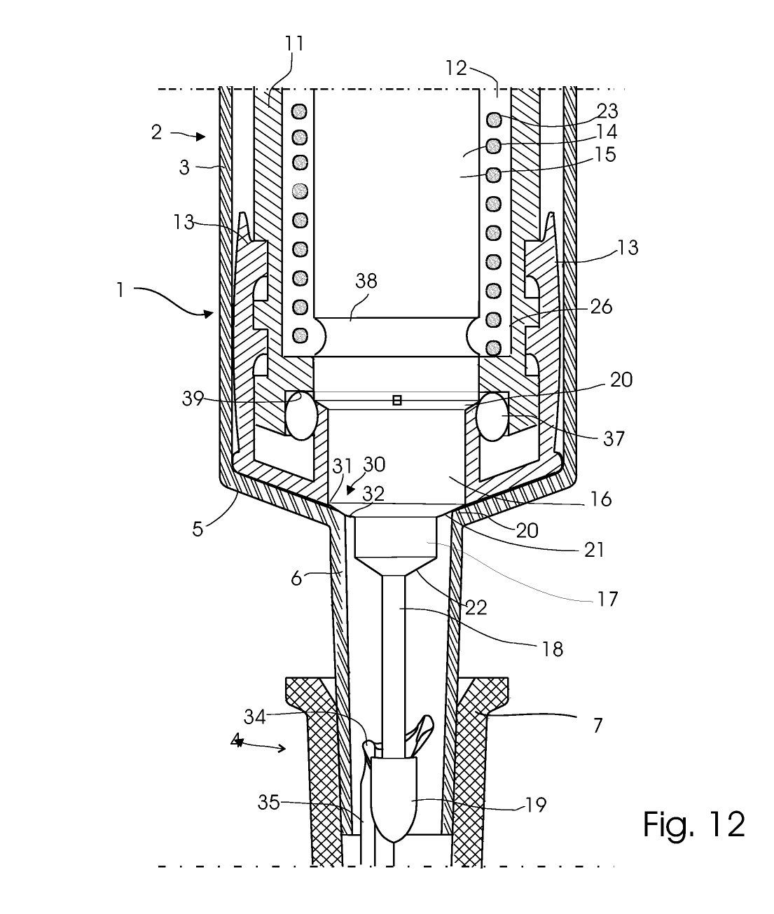

FIG. 12 shows the position of the retraction member immediately after the releasing of the retraction member where the hook 19 has passed the ring 34. The retraction member will then be retracted into the housing influenced by the spring power of the spring. The needle holder with the hypodermic needle will, during the continued retracting process, be coupled together with the retraction member and by this be pulled completely into the tubular housing and/or the hollow plunger.

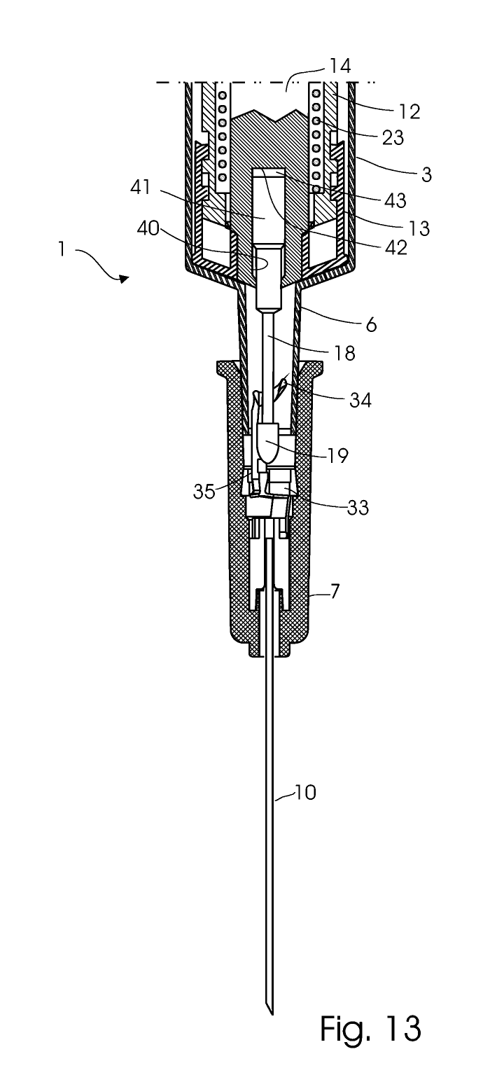

FIG. 13 shows an embodiment of the injection syringe shown in FIG. 1-12. For like parts same reference numerals are used.

The syringe is in this case equipped with a shock absorber means adapted for preventing the injection syringe to be affected by shocks during retracting of the hypodermic needle into the cavity of the plunger.

Said shock absorber means consists of a blind hole 40 extending into the retraction member 14 from the second releasing stop 32 on the retraction member 14 and of a piston 41 which is slidable placed in the blind hole 40 and connected to the fourth retraction member part 18.

FIG. 13 shows the position of the plunger 14 in which the injection operation is finished and the retraction operation is going to begin. The hook 19 has not yet engaged the ring 34 of the strap 35.

In the air space 43 between the blind hole 41 and the bottom 42 of the blind hole a quantity of air is confined.

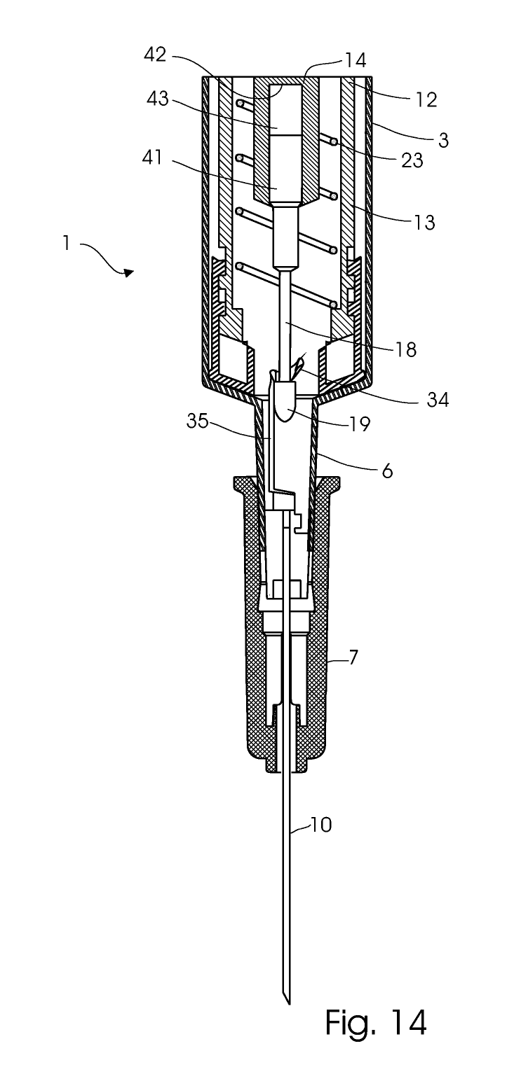

The retraction member 14 has, in FIG. 14, been released from the plunger 11 and is now on its way to the start position in the cavity 12 of the plunger 11. The hook 19 has engaged the ring 34 of the strap 35 whereby the retraction member 14 via the piston 41 pulls the hypodermic needle 10 out of the needle holder 33 in the cap 7.

The force needed for pulling the needle 10 out of the needle holder 33 tends to pull the sliding piston in the blind hole 40 in the opposite direction whereby the quantity of air confined in the blind hole 40 behind the piston 41 expands and builds up a negative pressure which counteracts said pulling force. The shown construction functions as a pneumatic shock absorber.

The clearance between the piston 41 and the wall of the blind hole 40 can according to the invention be of a size which is small enough to allow the necessary negative pressure in the confined air to build up during release of the hypodermic needle 10 from the needle holder 33.

The clearance shall on the other hand be large enough to allow air from the surroundings to ooze into the air space 43 for breaking down the negative pressure when the hypodermic needle has been released thereby avoiding that the piston 41 in the blind hole 40 acts as a pneumatic spring shocking the syringe at the end of the return stroke.

Alternatively, one or more through holes in the wall of the retraction member around the air space 43 can be used instead of the clearance.

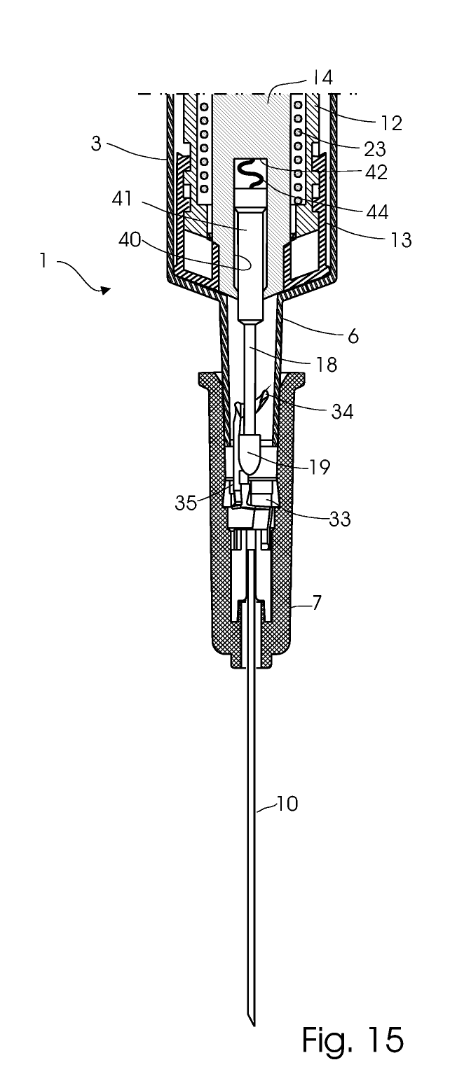

FIG. 15 shows a variant of the injection syringe shown in FIGS. 13 and 14. For like parts same reference numerals are used.

In this case an elastic strap 44 has been attached to the blind hole 40 at one end and to the piston 41 at the other end.

FIG. 15 shows the position of the plunger 14 in which the injection operation is finished and the retracting operation is going to begin. The hook 19 has not yet engaged the ring 34 of the strap 35.

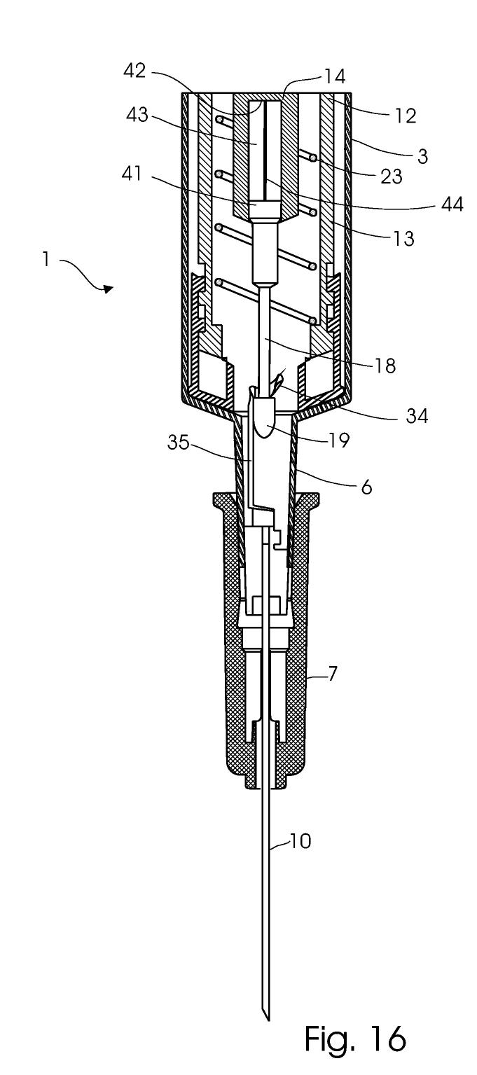

The retraction member 14 has in FIG. 16 been released from the plunger 11 and is now on its way to the start position in the cavity 12 of the plunger 11. The hook 19 has engaged the ring 34 of the strap 35 whereby the retraction member 14 via the piston 41 pulls the hypodermic needle 10 out of the needle holder 33 in the cap 7.

The force needed to pull the needle 10 out of the needle holder 33 tends to pull the sliding piston in the blind hole 40 into the opposite direction thereby stretching the elastic strap 44. The elastic force of the stretched strap will counteract the retraction force.

The desired shock absorbing effect can be performed by means of the elastic strap only. The clearance between the piston 41 and the wall of the blind hole 40 is in this case so large that a negative pressure is not allowed to build up in the air space 43.

The desired effect can, according to the invention, also be obtained by means of a combination of the shock absorber means shown in FIGS. 13 and 14 with the shock absorber means shown in FIGS. 15 and 16.

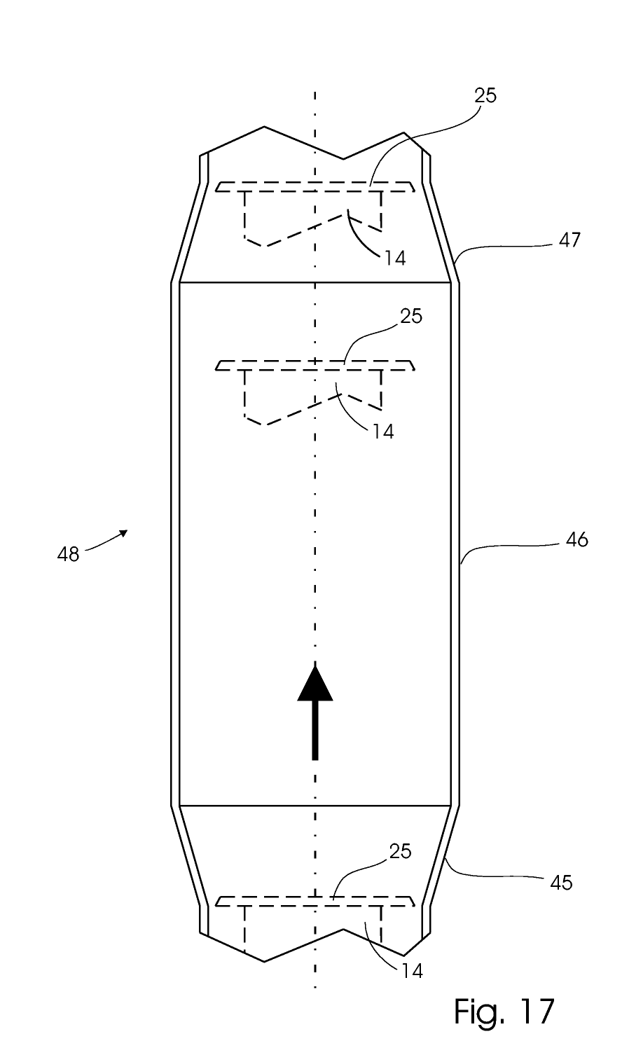

FIG. 17 shows, somewhat exaggerated, a fragment of a plunger 48 and the retraction member 14 in still another embodiment of the injection syringe shown in FIG. 1-12. For like parts same reference numerals are used.

The shown fragment of the plunger 48 is divided into a first, second and third section 45, 46 and 47.

The second section 46 has a diameter sufficiently large in relation to the diameter of the head 25 on the retraction member 14 to secure that the retraction member is not acted on by any significant air resistance when passing this section into the direction shown by the arrow.

The first and third sections 45 and 47 are tapering against the second section 46 from an inner diameter approximately corresponding to or slightly larger than the outer diameter of the retraction member head 25 to the same inner diameter as the second section 46. The difference between the outer diameter of the head 25 and the inner diameter of the second section 46 varies in dependency of parameters such as e.g. the size of the syringe and the characteristics of the spring.

The retraction member 14 therefore will pass the first section 45 into the direction of the arrow with a velocity increasing from a low velocity to a high velocity. The retraction member 14 will keep the high velocity during the subsequent passage of the second section. Then the large velocity will decrease to a low velocity again when the retraction member 14 finally is passing the third section 47.

Thereby advantageously is obtained that the retraction of the hypodermic needle 10 into the cavity 12 of the plunger 11 takes place in a very fast way and at the same time also so moderate that the syringe according to the invention is comfortable to operate for the operator, e.g. a doctor or nurse and the risk is reduced for unintentionally hitting and thereby harming somebody with the needle before fully retraction is obtained. Also, aerification of blood residues on the needle 10 is advantageously prevented.

The wall of the plunger 48 has in the embodiment shown in FIG. 17 the same thickness all along the length of the plunger.

The size of the outer diameter of the plunger is in a preferred embodiment (not shown) the same in each cross section along the length of the plunger while the size of the internal diameter varies in the same way as illustrated in FIG. 17 whereby also the wall thickness varies.

In another embodiment (not shown) is the size of the area of the cavity of plunger varied by means of at least one axially extending groove, which from the inside is formed in the wall of the plunger and has varying depth along the length of the plunger.

The embodiment shown in FIG. 17 is arranged for shock absorbing at both end of the return stroke. Within the scope of the invention can a plunger for the injection syringe according to the invention be arranged for shock absorbing in only one of the ends of the return stroke, par example the starting end.

1-20. (canceled)

21. An injection syringe comprising:
a tubular housing,
a hollow plunger disposed for reciprocating displacements, partly in the housing, between advanced and a retracted positions,
an elongated retraction member arranged for movement between advanced and retracted positions in relation to the plunger with at least a part of the retraction member placed inside the plunger, with the retraction member including a hook,
at least one spring acting with a spring power between the plunger and the retraction member,
a hypodermic needle displaceable between a first position wherein it is releasably mounted in the housing with a part of the needle protruding from the housing, and a second position in which the needle is retracted in the housing or the plunger,
a releasable retainer arrangement acting between the plunger and the retraction member for retaining the retraction member in its advanced position in relation to the plunger,
releasing means for releasing the retainer arrangement when the plunger is in or close to its advanced position, and
a coupling for connecting the retraction member and the needle during or immediately after releasing of the retainer arrangement,
a pneumatic shock absorber comprising a blind hole extending into the retraction member from a terminal surface opposite to the head on the retraction member, and a piston which is slidably arranged in the blind hole and connected to the retraction member.

22. The injection syringe according to claim 21, wherein the coupling comprises a first coupling part on the retraction member and a second coupling part on the needle.

23. The injection syringe according to claim 22, wherein the first coupling part comprises a hook and the second coupling part a ring arranged for allowing the hook to pass through in the direction of the needle but not in the opposite direction.

24. The injection syringe according to claim 23, wherein the hook is arranged at the end of a protruding pin of the retraction member and the ring is connected with the needle or a holder for the needle by means of at least one strap having a length that is longer than that of the hook.

25. The injection syringe according to claim 21, wherein the releasing means comprises a first releasing stop on the housing co-operating with a second releasing stop on the retraction member to prevent further advancing of the retraction member when the plunger is pushed forward in the housing to an intermediate position situated at a distance from the advanced position of the plunger, and the retainer arrangement is formed with a resistance against axial loadings which is lower than the reaction force acting between the first and second stop when pushing the plunger forward from the intermediate position with a predetermined force.

26. The injection syringe according to claim 21, wherein the retainer arrangement comprises a first retaining part on the plunger and a second retaining part on the retraction member.

27. The injection syringe according to claim 26, wherein the first or second retaining parts are deformable to such an extent that they are able to pass each other during movement.

28. The injection syringe according to claim 26, wherein the first retaining part is equipped with at least one first projection and the second retaining part with at least one second projection, with the at least one first and second projections overlapping each other radially and abutting each other influenced by the spring power of the at least one spring.

29. The injection syringe according to claim 28, wherein the at least first or second projections are breakable.

30. The injection syringe according to claim 21, wherein the tubular housing comprises a first tubular housing part accommodating the plunger, a second tubular housing part having a smaller diameter than the first housing part, and a transverse third housing part connecting the first and second housing parts.

31. The injection syringe according to claim 30, wherein the first releasing stop is formed on the third housing part and that the second releasing stop is an opposite shoulder on the retraction member.

32. The injection syringe according to claim 30, wherein the plunger at its end portion is equipped with a gasket for sealing the plunger in relation to the first tubular housing part.

33. The injection syringe according to claim 32, wherein the gasket is arranged for, in the advanced position of the retraction member, sealing the retraction member in relation to the plunger simultaneously with sealing the plunger in relation to the first tubular housing part.

34. The injection syringe according to claim 33, wherein the pneumatic shock absorber is a compressible gasket is arranged for abutting at least part of the inner side of the third housing part while pushing the plunger forward in the first housing part from at least the intermediate position of the plunger.

35. The injection syringe according to claim 34, wherein the gasket is made of an elastomer.

36. The injection syringe according to claim 34, wherein the gasket is an elastic strap attached to the blind hole at one end and to the piston at the other end.

37. A method for producing the injection syringe of claim 21, wherein the hollow plunger and the elongated retraction member are injection-molded simultaneously in such way that the first retaining part on the plunger and the second retaining part on the retraction member are integrally molded.
